(19) United States
(12) Patent Application Publication
(10) Pub. No.: US 2018/0104266 A1
(43) Pub. Date: Apr. 19, 2018

(54) SUGAR-BASED DISPERSION

(71) Applicant: Mary Kay Inc., Addison, TX (US)

(72) Inventor: James SWANZY, Arlington, TX (US)

(21) Appl. No.: 15/845,608

(22) Filed: Dec. 18, 2017

Related U.S. Application Data
(63) Continuation of application No. 13/320,138, filed on Nov. 11, 2011, now abandoned.
(60) Provisional application No. 61/388,803, filed on Oct. 1, 2010.

(51) Int. Cl. A61K 31/70; A61K 8/04; A61Q 19/00; A61K 8/60; A61K 8/31; A61K 8/92

(52) U.S. Cl. CPC A61K 31/70; A61K 2800/31; A61K 2800/28; A61K 8/044; A61K 8/922; A61Q 19/00; A61K 8/60; A61K 8/31

(57) ABSTRACT

Disclosed is a stable anhydrous dispersion comprising a dispersed sugar phase comprising granulated sugar and powdered sugar, wherein the dispersion comprises 55% to 75% by weight, based on the total weight of the composition, of a combination of granulated sugar and powdered sugar, and a continuous oil phase, wherein the dispersion comprises at least 20% by weight, based on the total weight of the composition, of an oil.

### CROSS REFERENCE TO RELATED APPLICATIONS

This application claims the benefit of U.S. Provisional Application No. 61/388,803, filed Oct. 1, 2010. The contents of the referenced application are incorporated by reference.

### BACKGROUND OF THE INVENTION

### A. Field of the Invention

The present invention relates generally to compositions that have granulated sugar, powdered sugar, and oil. In one aspect, the composition can be used to moisturize skin or exfoliate skin. In another aspect, the composition can be used as a cleanser to remove dirt, oil, grease, tars, from surfaces etc.

### B. Description of Related Art

Several skin moisturizing and/or exfoliating compositions are currently available. These compositions have various drawbacks ranging from unpleasant tactile properties (e.g., heavy, greasy, or sticky feel), instability issues, skin-irritation issues, or insufficient moisturization capabilities.

Further, cleansing compositions oftentimes have ingredients that can be caustic to the surfaces to be cleansed. For instance, many types of cleansers use surfactants, which can cause skin irritation.

### SUMMARY OF THE INVENTION

The present invention overcomes deficiencies in the art by providing a stable anhydrous dispersion that includes a dispersed sugar phase and a continuous oil phase. The dispersed sugar phase can include a combination of granulated sugar and powdered sugar. The continuous oil phase can include an oil (e.g., natural and/or synthetic oils) or a combination of oils. One unique aspect of this dispersion is that it can remain stable without using a surfactant (which is a known skin irritant). The dispersion can moisturize skin, exfoliate skin, and/or cleanse skin and other objects (e.g. articles of manufacture).

In one aspect of the present invention there is disclosed an anhydrous dispersion comprising a dispersed sugar phase comprising granulated sugar and powdered sugar, wherein the dispersion comprises 55% to 75% by weight, based on the total weight of the dispersion, of a combination of granulated sugar and powdered sugar, and a continuous oil phase, wherein the dispersion comprises at least 20% by weight, based on the total weight of the dispersion, of an oil. The weight ratio of granulated sugar to powdered sugar within the composition can be from 1:1 to 2:1, 1.1:1 to 2:1, 1.2:1 to 2:1, 1.3:1 to 2:1, 1.4:1 to 2:1, 1.5:1 to 2:1, 1.6:1 to 2:1, 1.7:1 to 2:1, 1.8:1 to 2:1, or 1.9:1 to 2:1. In certain aspects, the dispersion can include 30% to 45% w/w of granulated sugar or 35% to 40% w/w thereof. The dispersion can include 15% to 40% w/w of powdered sugar or 20% to 35% w/w thereof. The dispersion can include 20, 25, 30, 35, 40, 45, 50, 55, 60, 65, 70, 75, 80, or 85% weight of the total weight of the dispersion (w/w) or more or any range or integer derivable therein of a combination of granulated sugar and powdered sugar. In particular embodiments, the dispersion can include between 55% to 75% w/w of a combination of granulated sugar to powdered sugar or 60% to 70% of a combination thereof. The dispersion can also include 5, 10, 15, 20, 25, 30, 35, 40, 45, or 50% w/w or more of an oil or a mixture of oils. In particular aspects, the dispersion can include 15% to 45% w/w of an oil or a mixture of oils. In certain aspects, the oil or combination of oils can be mineral oil, plant-derived oils (e.g., safflower oil, sweet almond oil, sunflower oil, olive oil, apricot kernel oil, jojoba oil, etc.), triglycerides (e.g., caprylic/capric triglyceride, etc.), essential oils, synthetic oils, natural oils, silicone-based oils, etc. or any combination thereof. In certain aspects, the dispersion includes at least some mineral oil or triglyceride or a combination thereof. The granulated sugar can have a mean aperture (MA) equal to or greater than 200 μm or between 200 μm to 1000 μm. The granulated sugar can be extra-fine, fine, medium, coarse, or extra-course. The powdered sugar can have a particle size of 2×, 3×, 4×, 5×, 6×, 7×, 8×, 9×, 10×, 11×, 12×, 13×, or 14×. In other present invention can also include additional cosmetic and/or pharmaceutical ingredients disclosed throughout this specification and those known in the art. In certain aspects, the additional cosmetic ingredient can be a dye, a sunscreen agent, a moisturization agent, a thickening agent, a silicone containing compounds, an emulsifier, a structuring agent, an antioxidant, or a vitamin, or any combination thereof. In particular embodiments, the dispersion is anhydrous (i.e., includes no or an insubstantial amount of water (e.g., less than 1%, less than 0.5%, less than 0.1%, less than 0.05%, etc.). Also, the dispersion can exclude glycerol, which can have a tendency to cause the granulated and/or powdered sugar to clump together. Interestingly, the dispersion does not require the use of a gelling or thickening agent to help disperse the granulated sugar within the oil or mixture of oils. That is to say, the dispersion can remain stable without the use of a gelling or thickening agent. The dispersions can also remain stable without using a surfactant or an emulsifier. That is to say, the dispersions can be surfactant free of emulsifier free. Further, the dispersion can have pleasant tactile properties without the use of silicone containing compounds (e.g., cyclomethicone, dimethicone, etc.). That is to say, the dispersion can have a cosmetic or pharmaceutically elegant feel without the use of silicone containing compounds. Further, the granulated sugars can be infused with additional ingredients (e.g., dyes, plant extracts, etc.) to provide additional visual and/or therapeutic properties to the dispersion. The dispersion can also be free of preservatives such as parabens (e.g., methyl paraben, propyl paraben, etc.). The dispersions can also be those described in the example section of this specification, which is incorporated by reference. The dispersions of the present invention can also include a warming or cooling agent, which can provide an endothermic or exothermic effect when dissolved in water or from the water or moisture on the skin. Non-limiting examples of such warming or cooling ingredients include magnesium sulfate, calcium chloride, ammonium nitrate, etc. The dispersions can also include or be admixed with sodium chloride, silicas, aluminas, talc, and other minerals such as iron oxides, titanium dioxide, zinc oxide, etc.

Also disclosed is a method of exfoliating skin comprising topical application of any one of the dispersions disclosed throughout this specification to skin in need thereof and rinsing said dispersion from skin. The dispersion can be rinsed with water, alcohol, oil, etc. The granulated sugar can aid in the skin exfoliation process by aiding in the removal of dead skin cells from the skin's surface.

In another aspect, there is disclosed a method of moisturizing skin comprising topical application to skin in need thereof any one of the dispersion disclosed throughout this specification. The dispersion can be applied to dry skin, flaky skin, chapped skin, cracked skin, etc.

A further embodiment includes a method of cleansing skin comprising topical application to skin in need thereof any one of the dispersions disclosed throughout this specification followed by rinsing the dispersion from skin. The dispersion can be rinsed with water, alcohol, oil etc. The dispersion can be applied to skin having dirt, oil, sebum, tar, or grease on said skin, with the result being that said dirt, oil, tar, sebum, or grease is removed from said skin. In certain aspects, the dispersion do not include traditional cleansing agents such as surfactants, which can be useful in reducing skin irritation.

It was also discovered that the dispersions of the present invention are capable of removing unwanted substances from articles of manufacture (e.g., cars, walls, toys, dishware, windows, etc.). The dispersion can be applied to an article of manufacture followed by rinsing with water, alcohol, oil, etc. The unwanted substance can be oil, grease, dirt, paint (e.g., oil-based paint or latex-based paint), tar, etc.

In yet another embodiment, there is a disclosed a method of treating or preventing a skin condition comprising topically applying any one of the dispersions disclosed throughout the specification to skin in need thereof. Non-limiting examples of such skin conditions include dry, cracked, or flaky skin (e.g., facial, scalp, hand, elbow, feet, heel, and other portions of skin that have a tendency to dry flake, or crack). Other conditions include fine lines or wrinkles, inflamed skin, erythemic skin, dead skin, sunburned skin, pruritus, spider veins, lentigo, age spots, senile purpura, keratosis, melasma, blotches, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions. The skin can be facial skin or non-facial skin (e.g., arms, legs, hands, chest, back, feet, etc.). The method can further comprise identifying a person in need of skin treatment. The person can be a male or female. The age of the person can be at least 1, 2, 3, 4, 5, 6, 7, 8, 9, 10, 15, 20, 25, 30, 35, 40, 45, 50, 55, 60, 65, 70, 75, 80, 85, 90, 95, or more years old, or any range derivable therein. The method can also include topically applying an amount effective to: increase the stratum corneum turnover rate of the skin; increase collagen synthesis in fibroblasts; increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, langerhans cells, etc.) which will reduce or prevent oxidative damage to the skin, cellular, proteins, and lipids); inhibit melanin production in melanocytes; reduce or prevent oxidative damage to skin (including reducing the amount lipid peroxides and/or protein oxidation in the skin).

The dispersions disclosed throughout this specification can be used on facial skin, and/or body skin (e.g., hands, arms, chest, abdomen, upper and lower back, legs, buttocks, feet, etc.).

In still another embodiment, there is disclosed a method of using any one of the aforementioned dispersions to treat wounds (e.g., bed sores, diabetic ulcers, surgical incisions, skin burns, scratches, abrasions, etc.). The dispersions are particularly suited for the wound environment due to their sugar content, which can be used to treat wounds and speed up the wound healing process, and the fact that the dispersion does not require caustic materials to remain stable (e.g., surfactants and other cleansing agents). In this sense, an all natural product can be used to treat wounds safely and effectively.

Kits that include the dispersions of the present invention are also contemplated. In certain embodiments, the dispersion is comprised in a container. The container can be a bottle, dispenser, or package. The container can dispense a pre-determined amount of the dispersion. In certain aspects, the dispersions is dispensed in a spray, dollop, or liquid. The container can include indicia on its surface. The indicia can be a word, an abbreviation, a picture, or a symbol.

Also contemplated is a product comprising a dispersion of the present invention. In non-limiting aspects, the product can be a cosmetic product. The cosmetic product can be those described in other sections of this specification or those known to a person of skill in the art. Non-limiting examples of products include an exfoliation product, a moisturizer, a cream, a lotion, a skin softener, a night cream, a cleanser, a toner, a sunscreen, a mask, an anti-aging product, etc.

It is contemplated that any embodiment discussed in this specification can be implemented with respect to any method or dispersion of the invention, and vice versa. Furthermore, dispersions of the invention can be used to achieve methods of the invention.

In one embodiment, the dispersions of the current invention are pharmaceutically elegant. “Pharmaceutically elegant” describes a dispersion that has particular tactile properties which feel pleasant on the skin (e.g., compositions that are not too watery or greasy, dispersions that have a silky texture, dispersions that are non-tacky or sticky, etc.). Pharmaceutically elegant can also relate to the creaminess or lubricity properties of the dispersion or to the moisture retaining properties of the dispersion.

Granulated sugar means sugar having a mean aperture (MA) of about equal to or greater than 200 μm. In particular embodiments, the mean aperture ranges from 200 μm to 1000 μm. In even more particular embodiments, the mean aperture is about 600 μm, with at least 80% product within 250 μm to 1000 μm.

Powdered sugar is sugar that has a mean aperture (MA) of less than 200 μm. In certain aspects, the average particle size is between 30 μm to 150 μm, as explained by U.S. Pat. No. 5,344,664, which is incorporated by reference. In particular aspects, the powdered sugar can be 10×, which has an approximate mean particle size of 40 μm, with 1% of the particles being no larger than 150 μm and 6% of the particles being no larger than 75 μm, as explained in U.S. Pat. No. 6,123,980, which is incorporated by reference.

“Keratinous tissue” includes keratin-containing layers disposed as the outermost protective covering of mammals and includes, but is not limited to, skin, hair and nails.

“Topical application” means to apply or spread a composition onto the surface of keratinous tissue. “Topical skin composition” and “dispersion” include compositions suitable for topical application on keratinous tissue. Such compositions are typically dermatologically-acceptable in that they do not have undue toxicity, incompatibility, instability, allergic response, and the like, when applied to skin. Dispersions of the present invention can have a selected viscosity to avoid significant dripping or pooling after application to skin.

“Exfoliating” means to remove dead or excess skin layers or cells from the surface of the skin.

“Surfactant-Free” means that the dispersion is surfactantless or free of surfactants. Surfactants include ingredients that have the ability to lower the surface tension of water or to reduce the interfacial tension between two immiscible substances. They are frequently classified as amphoteric, anionic, cationic, or nonionic surfactants.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The dispersions of the present invention can comprise, consist essentially of, or consist of the claimed ingredients. In one aspect, dispersions consisting essentially of the claimed ingredients excludes ingredients that would materially affect the stability of the dispersion (e.g., cause the dispersed phase to separate from the continuous phase or cause the sugar phase to coalesce into hard clumps).

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

In today's image conscious society, people are continually looking for a product that can improve the visual appearance of their skin. For instance, symptoms associated with dry skin (e.g., flaky skin, dried or rough tactile quality, cracked skin, dehydrated skin, itchy skin, or red or erythemic skin) is associated with unattractive skin. Similarly, un-cleansed skin can be unsightly and can also lead to skin infections, lesions, pimples, acne, etc. Also, older aged, weathered, or damaged skin can be aesthetically unpleasing, and can be rejuvenated through exfoliation of the skin.

The inventor discovered that a combination of granulated sugar, powdered sugar, and an oil or a mixture of oils produces a stable and multi-functional dispersion that has the ability to moisturize skin, cleanse skin, and exfoliate skin. Unlike other products on the market, the dispersions of the present invention have a relatively high content of natural ingredients, and in some instances, are completely made up of natural ingredients (e.g. sugar and natural oils derived from fruits, trees, bushes, vegetables, etc.). Further, the dispersions are storage stable in that the granulated sugar remains suspended or dispersed throughout the oil or mixture of oils with minimal to no coalescence of said granulated sugar during storage. A benefit of such stability is that the end user of the product does not have to mix or shake the product before each use. This results in a consistent product being dispensed from the container, whereas compositions that have active ingredients that settle or coalesce can often times be widely inconsistent with each use (e.g., the dispensed product may have more or less of the active ingredient each time it is dispensed from the container).

These and other non-limiting aspects of the present invention are described in further detail below.

### A. Granulated and Powdered Sugars

Descriptions of granulated and powdered sugars can be found in Beet-Sugar Handbook (2007), by Mosen Asadi, PhD., and Sugar, A User's Guide to Sucrose (1990), by Neil L. Pennington and Charles W. Baker, both of which are incorporated into this specification by reference. In summary, the size of sugar crystals (particle size) of granulated sugar ranges from a MA of about 200 μm to greater than 1000 μm, with MA referring to the size of the screen opening, on which 50% of the sugar sample stays and 50% passes through. Granulated sugars are typically categorized into extra-fine (MA of about 200 μm), fine (MA of about 300 μm), medium (MA of about 500 μm), coarse (MA of about 1000 μm), and extra-course (MA above 1000 μm). Each category of granulated sugar are commercially available from a wide range of sources (see, e.g., standard grocery stores in the United States such as H-E-B in Dallas, Tex.). All sizes of granulated sugar are contemplated.

Powdered sugar is ground granulated sugar that can also include anticaking agents (e.g., starch) to prevent clumping or lumping of the powdered sugar. Powdered sugar has a smaller particle size when compared with granulated sugar. It is commercially available from a wide range of sources (see, e.g., standard grocery stores in the United States such as H-E-B in Dallas, Tex.). Typically, powdered sugar is categorized and sold as 2X, 3X, 4X, 5X, 6X, 7X, 8X, 9X, 10X, 11X, 12X, 13X, and 14X powdered sugar (the higher the number preceding the X, the finer the particles). The particle size of powdered sugar typical falls within the range of an average particle size of 30 μm to 150 μm. In particular embodiments, 10× is used due to it being a common product in grocery stores.

### B. Oil

In addition to skin moisturization and fragrance benefits, oils can be used to suspend the granulated sugar. Natural and synthetic-based oils and mixtures thereof can be used. Non-limiting examples of oils that can be used in the context of the present invention can be found in the CTFA International Cosmetic Ingredient Dictionary and Handbook (2004 and 2008, editions). For instance, natural oils include oils derived from herbs, flowers, trees, vegetables, fruit, and other plants. Such oils can be extracted by several method known to those of skill in the art (e.g., steam distilled, enfleurage (i.e., extraction by using fat), maceration, solvent extraction, or mechanical pressing). Non-limiting examples of such oils include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang, jojoba oil, apricot kernel oil, safflower oil, sunflower oil, almond oil, and/or olive oil, or any mixture thereof. Non-limiting examples of oils not derived from plants that can be used include mineral oil, triglycerides, butter, fat, fatty acids, and/or silicone-based oils or silicone containing compounds. In certain aspects, the oil can be olive oil, shea butter, or cocoa butter. Other types of synthetic-based oils that can be used include triglycerides that have acid functional groups such as Cl-C30 chains that are saturated, un-saturated, or poly-unsaturated, or aromatic acids (e.g., benzoic acid). Any mixture of natural or synthetic-based oils can be used.

In certain embodiments, the oils that can be used can be natural-based oils, which can result in a dispersion that includes all natural products or a dispersion that does not include any synthetic products. In one instance, natural-based oils are those that have been described as such by organizations such as COSMOS (Cosmetics Organic and Natural Standard), Natural Products Association (NPA) (Natural Products Association Standard and Certification for Personal Care Products), COLIPA Guidelines, Unitis (European Natural and Organic Standards for Cosmetic Ingredients), etc.

### C. Method of Making the Dispersion

In addition to the methods disclosed in the Examples section of this specification, another non-limiting method for making a dispersion of the present invention includes: (1) obtain granulated and powdered sugar (any brands sold in grocery stores can be used-the Examples used C&H Pure Cane granulated sugar and C&H Pure Cane powdered/confectioners sugar, both of which were purchased at a Walmart in Dallas Tex.); (2) obtain baby oil (any oil can be used). Baby oil is commercially available at grocery stores and is typically used on skin, which makes for an simple and economical way to make a non-limiting dispersion of the present invention); (3) mix approximately ½ cup of granulated sugar and about ¼ to ⅓ cup of powdered sugar in a container; (4) add baby oil into the sugar and mix until a desired consistency is reached and the dispersion is stable (note that stability can be visually inspected by seeing the granulated sugar sufficiently dispersed in the baby oil without coalescence of the granulated sugar.). The dispersion is ready to be used and can be used in any manner described throughout this specification.

### D. Additional Ingredients

Compositions of the present invention can include additional ingredients. Non-limiting examples of additional ingredients include cosmetic ingredients (both active and non-active) and pharmaceutical ingredients (both active and non-active).
1. Cosmetic Ingredients

The CTFA Handbook describes a wide variety of non-limiting cosmetic ingredients that can be used in the context of the present invention. Examples of these ingredient classes include: alcohols benzoate, fragrances (artificial and natural), non-aqueous extracts, pigments (natural and synthetic), vitamins (e.g., A, B, C, D, E, and K), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, emulsifiers, stabilizers, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (“PABA”) and corresponding PABA derivatives, titanium dioxide, zinc oxide, etc.), essential oils, trace metals (e.g., zinc, calcium and selenium), anti-irritants (e.g., steroids and non-steroidal anti-inflammatories), botanical extracts (e.g., aloe vera, chamomile, cucumber extract, ginkgo biloba, ginseng, and rosemary), anti-microbial agents, antioxidants (e.g., BHT), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., propylene glycol, butylene glycol, pentylene glycol, sorbitol, urea, and manitol), exfoliants (e.g., alpha-hydroxyacids, and beta-hydroxyacids such as lactic acid, glycolic acid, and salicylic acid; and salts thereof) waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate), thickening agents (e.g., substances which that can increase the viscosity of a composition such as carboxylic acid polymers, crosslinked polyacrylate polymers, polyacrylamide polymers, polysaccharides, and gums), and silicone containing compounds (e.g., silicone oils and polyorganosiloxanes). The following provides specific non-limiting examples of some of the additional ingredients that can be used with the compositions of the present invention.

a. UV Absorption or Sunscreen Agents

UV absorption or sunscreen agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutyiphenol, and bis-ethylhexyloxyphenol methoxyphenyltriazine, 4-methylbenzylidenecamphor, and isopentyl 4-methoxycinnamate. Non-limiting examples of physical sunblocks include, kaolin, talc, petrolatum and metal oxides (e.g., titanium dioxide and zinc oxide).Compositions of the present invention can have UVA and UVB absorption properties. The compositions can have an sun protection factor (SPF) of 2, 3, 4, 56, 7, 8, 9, 10, 11, 12, 13, 14, 15, 20, 25, 30, 35, 40, 45, 50, 55, 60, 70, 80, 90 or more, or any integer or derivative therein.

b. Moisturizing Agents

Non-limiting examples of moisturizing agents that can be used with the compositions of the present invention include glycerin, glycols, amino acids, chondroitin sulfate, diglycerin, erythritol, fructose, glucose, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, acrylates/C10-30 alkyl acrylate crosspolymer, acrylates copolymer, alanine, algae extract, aloe barbadensis, aloe-barbadensis extract, aloe barbadensis gel, althea officinalis extract, aluminum starch octenylsuccinate, aluminum stearate, apricot (prunus armeniaca) kernel oil, arginine, arginine aspartate, arnica montana extract, ascorbic acid, ascorbyl palmitate, aspartic acid, avocado (persea gratissima) oil, barium sulfate, barrier sphingolipids, butyl alcohol, beeswax, behenyl alcohol, beta-sitosterol, BHT, birch (betula alba) bark extract, borage (borago officinalis) extract, 2-bromo-2-nitropropane-1,3-diol, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrageenan (chondrus crispus), carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays)oil, fatty acids, decyl oleate, dextrin, diazolidinyl urea, dimethicone copolyol, dimethiconol, dioctyl adipate, dioctyl succinate, dipentaerythrityl hexacaprylate/hexacaprate, DMDM hydantoin, DNA, erythritol, ethoxydiglycol, ethyl linoleate, eucalyptus globulus oil, evening primrose (oenothera biennis) oil, fatty acids, tructose, gelatin, geranium maculatum oil, glucosamine, glucose glutamate, glutamic acid, glycereth-26, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, honey, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, imidazolidinyl urea, iodopropynyl butylcarbamate, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, magnesium stearate, magnesium sulfate, maltitol, matricaria (chamomilla recutita) oil, methyl glucose sesquistearate, methylsilanol PCA, microcrystalline wax, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rosemary (rosmarinus officinalis) oil, rose oil, safflower (carthamus tinctorius) oil, sage (salvia officinalis) oil, salicylic acid, sandalwood (santalum album) oil, serine, serum protein, sesame (sesamum indicum) oil, silk powder, sodium chondroitin sulfate, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopheryl linoleate, tribehenin, tridecyl neopentanoate, tridecyl stearate, triethanolamine, tristearin, urea, vegetable oil, water, waxes, wheat (triticum vulgare) germ oil, and ylang ylang (cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocophersolan, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agents, in certain aspects, assist in providing rheological characteristics to the composition to contribute to the composition's stability. In other aspects, structuring agents can also function as an emulsifier or surfactant. Non-limiting examples of structuring agents include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Surfactants/Emulsifiers

In some non-limiting aspects, the compositions can include one or more surfactants/emulsifiers. Surfactants/emulsifiers can reduce the interfacial tension between phases and improve the formulation and stability of an emulsion. The surfactants/emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

f. Thickening Agents

Thickening agents, including thickener or gelling agents, include substances that can increase the viscosity of a composition. Thickeners include those that can increase the viscosity of a composition without substantially modifying the efficacy of the active ingredient within the composition. Thickeners can also increase the stability of the compositions of the present invention.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, 4^{th} Ed., 1991). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

g. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof.

h. Skin Lightening Agents

Non-limiting examples of skin lightening agents that can be used in the context of the present invention include dipotassium glycyrrhizate, ascorbyl glucoside, niacinamide, hydroquinone, or combination thereof.

i. Silicone Containing Compounds

Silicone containing compounds can provide a silky, non-oily feel to topical skin care compositions. In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

In one non-limiting aspect of the present invention, the compositions can include about 0.1 to 2% w/w of a silicone containing compound. In particular aspects, the inventor discovered that dimethicone in combination with the cationic surfactant, the moisturizing agents, and the relatively high amount of water worked well in the context of the present invention.
2. Pharmaceutical Ingredients

Pharmaceutical ingredients are also contemplated as being useful with the emulsion compositions of the present invention. Non-limiting examples of pharmaceutical ingredients include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### E. Combinations and Amounts of Ingredients

It is contemplated that the compositions of the present invention can include the granulated sugar, the powdered sugar, and the oil or mixture of oils. The compositions can also include additional ingredients described throughout this specification. The concentrations of the granulated sugar, the powdered sugar, and the oil or mixture of oils, or any additional ingredients can vary. In non-limiting embodiments, for example, the compositions can include in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or more, or any range or integer derivable therein, of at least one of, any combination of, or all of the granulated sugar, the powdered sugar, the oil or mixture of oils, and any additional ingredient disclosed throughout this specification. In non-limiting aspects, the percentage of such ingredients can be calculated by weight or volume of the total weight of the compositions. The concentrations can vary depending on the desired effect of the compositions or on the product into which the compositions are incorporated.

### F. Composition Vehicles

The compositions of the present invention can be formulated into all types of vehicles. Non-limiting examples of suitable vehicles include dispersions, emulsions, creams, lotions, ointments, pastes, milks, liquids, solid forms. Variations and other appropriate vehicles will be apparent to the skilled artisan and are appropriate for use in the present invention. In certain aspects, the concentrations and combinations of the ingredients can be selected in such a way that the combinations are chemically compatible and do not form complexes which precipitate from the finished product.

### G. Products

The compositions of the present invention can be incorporated into cosmetic products, food-based products (e.g., fortified water, energy drinks, nutritional drinks, vitamins, supplements, solid foods), pharmaceutical products, etc. Non-limiting examples of cosmetic products include exfoliating products, skin moisturization products, cleansing products, body washing products, shampoos, creams, lotions, sunless skin tanning products, fingernail products, moisturizing creams, skin creams and lotions, softeners, day lotions, ointments, foundations, night creams, lipsticks and lip balms, cleansers, toners, masks, deodorants, antiperspirants, shaving-related products (e.g., creams, “bracers” and aftershaves), pre-moistened wipes and washcloths, tanning lotions, bath products such as oils, foot care products such as powders and sprays, skin colorant and make-up products such as foundations, blushes, rouges eye shadows and lines, lip colors and mascaras, baby products (e.g., baby lotions, oils, shampoos, powders and wet wipes), and skin or facial peel products. Additionally, the cosmetic products can be formulated as leave-on or rinse-off products.

### H. Kits

Kits are also contemplated as being used in certain aspects of the present invention. For instance, a composition of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of a composition. In other embodiments, the container can be squeezed (e.g., metal, laminate, or plastic tube) to dispense a desired amount of the composition. The composition can be dispensed as a spray, foam, an aerosol, a liquid, a fluid, or a semi-solid. The containers can have spray, pump, or squeeze mechanisms. A kit can also include instructions for using the kit and/or compositions. Instructions can include an explanation of how to apply, use, and maintain the compositions.

### EXAMPLES

The following examples are included to demonstrate certain non-limiting aspects of the invention. It should be appreciated by those of skill in the art that the techniques disclosed in the examples which follow represent techniques discovered by the inventors to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Stability and Tactile Property Testing

It was discovered that the ratio of the total amount of oil to the total sugar amount in the formulations of the present invention affects both the consistency and stability of the formulations. The following four formulations illustrate this.

TABLE 1*

| Phase A | Ingredient | Amount by weight (grams) |
|---|---|---|
| A | Granulated sugar** | 25 |
| | Powdered sugar*** | 25 |
| | Plant extracts | 0.5 |
| B | Mineral oil, light | 44.5 |
| | C12-15 Alcohols benzoate | 5.0 |
| | Total | 100 |

*Formulation was prepared as follows (all steps were performed at room temperature, i.e., approx. 20-25° C., and without any heating or cooling apparatuses): (1) weighed ingredients; (2) mixed the granulated sugar, powdered sugar, and plant extracts in a container and with a spatula until mixture was thoroughly blended (sugar phase); (3) in a separate container, mixed the C12-15 Alcohols benzoate and mineral oil (oil phase); (4) added the oil phase to the sugar phase and mixed with a spatula until the granulated sugar was dispersed throughout the oil phase.
**Granulated sugar was C&H Pure Cane Sugar (Granulated White) and was purchased at Walmart in Dallas Texas. This is the same sugar used in the formulations described in Tables 2-16.
***Powdered sugar was C&H Pure Cane Sugar (Confectioners Powdered) and was purchased at Walmart in Dallas Texas. This is the same sugar used in the formulations described in Tables 2-16.

The Table 1 formulation, which includes 50% total sugar and 44.5% total oil, had a thin tactile feel and separated shortly after being prepared and was therefore not stable.

In particular, the sugar phase separated from the oil phase. Note that the C12-15 Alcohols benzoate ingredient was added solely to impart a skin moistuization effect to the formulation (in addition to the natural skin moisturization effect of sugar) and the plant extracts were added solely to provide additional skin efficacy benefits to the formulation. That is, the addition of the C12-15 Alcohols benzoate and plant extract ingredients in the amounts added did not play a role in the stability/instability of the formulation. In addition to these ingredients, it is contemplated that the additional ingredients described throughout the specification can be incorporated into the formulation.

TABLE 2*

| | | Amount by weight |
|---|---|---|
| Phase | Ingredient | (grams) |
| A | Granulated sugar | 35 |
| | Powdered sugar | 25 |
| | Plant extracts | 0.2 |
| B | Mineral oil, light | 34.8 |
| | C12-15 Alcohols benzoate | 5.0 |
| | Total | 100 |

*Formulation was prepared in the same manner as the Table 1 formulation.

The Table 2 formulation, which includes 60% total sugar and 34.8% total oil, had a lotion-like tactile feel and remained sufficiently stable after being prepared (i.e., the granulated sugar remained substantially suspended and dispersed within the oil phase when stored at room temperature (approximately 20-25° C.) and when heat was applied).

TABLE 3*

| | | Amount by weight |
|---|---|---|
| Phase | Ingredient | (grams) |
| A | Granulated sugar | 35 |
| | Powdered sugar | 35 |
| | Plant extracts | 0.2 |
| B | Mineral oil, light | 24.8 |
| | C12-15 Alcohols benzoate | 5.0 |
| | Total | 100 |

*Formulation was prepared in the same manner as the Table 1 formulation.

The Table 3 formulation, which includes 70% total sugar and 24.8% total oil, had a creamy-like tactile feel and remained sufficiently stable after being prepared (i.e., the granulated sugar remained substantially suspended and dispersed within the oil phase when stored at room temperature (approximately 20-25° C.) and when heat was applied).

TABLE 4*

| | | Amount by weight |
|---|---|---|
| Phase | Ingredient | (grams) |
| A | Granulated sugar | 40 |
| | Powdered sugar | 30 |
| | Plant extracts | 0.2 |
| B | Mineral oil, light | 24.8 |
| | C12-15 Alcohols benzoate | 5.0 |
| | Total | 100 |

*Formulation was prepared in the same manner as the Table 1 formulation.

The Table 4 formulation, which includes 70% total sugar and 24.8% total oil, also had a creamy-like tactile feel and remained sufficiently stable after being prepared (i.e., the granulated sugar remained substantially suspended and dispersed within the oil phase when stored at room temperature (approximately 20-25° C.) and when heat was applied). The difference between the Table 3 and Table 4 formulations is that the Table 4 formulation included more granulated sugar.

The Table 1-4 formulations suggest that the total amount of the sugar mixture compared with the total amount of the oil can affect the stability and tactile feel of the formulation. A formulation having 50% total sugar produced an unstable dispersion in that the sugar phase separated from the oil phase shortly after being prepared. By increasing the total content to 60% and to 70%, the stability of the formulation became stable. Also, stability of the dispersion was obtained without using a surfactant/emulsifier. As shown below, stability can be achieved by going as low as 55% w/w total sugar and as high as 75% w/w total sugar. Below and above this range results in inadequate products.

It was also surprisingly discovered that the addition of glycerol or glycerin in amounts of 5% w/w and greater negatively affected the dispersion in that it resulted in clumping of the sugar into hard lumps (visual inspection). It was also found that the presence of mineral oil surprisingly (1) increased the spreadability (i.e., low drag) of the dispersion onto a surface when compared with other oils (data not shown) and (2) increased stability of the dispersion when compared with other oils (data not shown).

The presence of granulated sugar in the dispersion allows for skin exfoliation. Both the granulated and confectioner sugars hydrate and moisturize the skin (data not shown). Further, confectioner sugar allows for the dispersion to appear as a cream or a lotion (visual inspection).

### Example 2

### Non-Limiting Product Formulations

Non-limiting product formulations are provided in the following Tables These formulations remained stable after being prepared in that the sugar phase remained substantially suspended within the oil phase when stored at room temperature (approximately 20-25° C.) and when heat was applied. The formulations were prepared as described in Table 1 (i.e., sugar phase is mixed in a separate container from the oil phase followed by subsequent mixing of the two phases at room temperature and with a spatula until the dispersion is formed).

TABLE 5*

| | | Amount by weight |
|---|---|---|
| Phase | Ingredient | (grams) |
| A | Granulated sugar | 40 |
| | Powdered sugar | 30 |
| | Plant extracts | 0.2 |
| B | C12-15 Alcohols benzoate | 5.0 |
| | Mineral oil, light | 21.8 |
| | Safflower oil | 0.5 |
| | Sweet almond oil | 0.5 |
| | Sunflower oil | 0.5 |
| | Jojoba Oil | 0.5 |
| | Fragrance | 1.0 |
| | Total | 100 |

*product has a creamy-like tactile feel.

TABLE 6*

| | | Amount by weight |
|---|---|---|
| Phase | Ingredient | (grams) |
| A | Granulated sugar | 40 |
| | Powdered sugar | 30 |
| | Plant extracts | 0.2 |
| B | C12-15 Alcohols benzoate | 5.0 |
| | Mineral oil, light | 21.8 |
| | Safflower oil | 0.5 |
| | Sweet almond oil | 0.5 |
| | Sunflower oil | 0.5 |
| | Jojoba Oil | 0.5 |
| | Fragrance | 0.2 |
| | Total | 100 |

*product has a creamy-like tactile feel.

TABLE 7*

| | | Amount by weight |
|---|---|---|
| Phase | Ingredient | (grams) |
| A | Granulated sugar | 35 |
| | Powdered sugar | 20 |
| | Plant extracts | 0.25 |
| B | C12-15 Alcohols benzoate | 5.0 |
| | Mineral oil, light | 41.8 |
| | Safflower oil | 0.5 |
| | Sweet almond oil | 0.5 |
| | Sunflower oil | 0.5 |
| | Jojoba Oil | 0.5 |
| | Fragrance | 0.1 |
| | Total | 105 |

*product has a lotion-like tactile feel.

TABLE 8*

| | | Amount by weight |
|---|---|---|
| Phase | Ingredient | (grams) |
| A | Granulated sugar | 35 |
| | Powdered sugar | 35 |
| B | Mineral oil, light | 15 |
| | Neutrogena Body Oil** | 15 |
| | Total | 100 |

*product has a creamy-like tactile feel.
**Neutrogena Body Oil is commercially available from Neutrogena.com. This product contains Isopropyl Myristate, Sesame Seed Oil (Sesamum Indicum), PEG 40 Sorbitan Peroleate, Propylparaben, BHT

TABLE 9*

| | | Amount by weight |
|---|---|---|
| Phase | Ingredient | (grams) |
| A | Granulated sugar | 40 |
| | Powdered sugar | 30 |
| | Plant extracts | 0.8 |
| B | Mineral oil, light | 20.2 |
| | C12-15 Alcohols Benzoate | 5 |
| | Safflower oil | 0.5 |
| | Sweet almond oil | 0.5 |
| | Sunflower oil | 0.5 |
| | Jojoba oil | 0.5 |
| | Fragrance | 2 |
| | Total | 100 |

*product has a creamy-like tactile feel.

TABLE 10*

| | | Amount by weight |
|---|---|---|
| Phase | Ingredient | (grams) |
| A | Granulated sugar | 40 |
| | Powdered sugar | 30 |
| | Plant extracts | 0.4 |
| B | Mineral oil, light | 21.6 |
| | C12-15 Alcohols Benzoate | 5 |
| | Safflower oil | 0.5 |
| | Sweet almond oil | 0.5 |
| | Sunflower oil | 0.5 |
| | Jojoba oil | 0.5 |
| | Fragrance | 1 |
| | Total | 100 |

*product has a creamy-like tactile feel.

TABLE 11*

| | | Amount by weight |
|---|---|---|
| Phase | Ingredient | (grams) |
| A | Granulated sugar | 40 |
| | Powdered sugar | 30 |
| | Plant extracts | 0.4 |
| B | Mineral oil, light | 11.6 |
| | C12-15 Alcohols Benzoate | 5 |
| | Safflower oil | 3 |
| | Sweet almond oil | 3 |
| | Sunflower oil | 3 |
| | Jojoba oil | 3 |
| | Fragrance | 1 |
| | Total | 100 |

*product has a creamy-like tactile feel.

TABLE 12*

| | | Amount by weight |
|---|---|---|
| Phase | Ingredient | (grams) |
| A | Granulated sugar | 35 |
| | Powdered sugar | 25 |
| | Plant extracts | 0.4 |
| B | Mineral oil, light | 17.6 |
| | C12-15 Alcohols Benzoate | 5 |
| | Safflower oil | 4 |
| | Sweet almond oil | 4 |
| | Sunflower oil | 4 |
| | Jojoba oil | 4 |
| | Fragrance | 1 |
| | Total | 100 |

*product has a lotion-like tactile feel.

TABLE 13*

| | | Amount by weight |
|---|---|---|
| Phase | Ingredient | (grams) |
| A | Granulated sugar | 40 |
| | Powdered sugar | 30 |
| | Aloe extract | 2 |
| B | Caprylic/Capric Triglyceride | 7 |
| | Safflower oil | 7 |
| | Olive oil | 7 |
| | Sunflower oil | 7 |
| | Total | 100 |

*product has a creamy-like tactile feel.

TABLE 14*

| | | Amount by weight |
|---|---|---|
| Phase | Ingredient | (grams) |
| A | Granulated sugar | 40 |
| | Powdered sugar | 22 |
| B | Caprylic/Capric Triglyceride | 15.5 |
| | Safflower oil | 6 |
| | Olive oil | 10 |
| | Sunflower oil | 6 |
| | Fragrance | 0.5 |
| | Total | 100 |

*product has a lotion-like tactile feel.

TABLE 15*

| | | Amount by weight |
|---|---|---|
| Phase | Ingredient | (grams) |
| A | Granulated sugar | 40 |
| | Powdered sugar | 31 |
| B | Caprylic/Capric Triglyceride | 13.5 |
| | Safflower oil | 5 |
| | Olive oil | 5 |
| | Sunflower oil | 5 |
| | Fragrance | 0.5 |
| | Total | 100 |

*product has a creamy-like tactile feel.

TABLE 16*

| | | Amount by weight |
|---|---|---|
| Phase | Ingredient | (grams) |
| A | Granulated sugar | 40 |
| | Powdered sugar | 30 |
| B | Mineral oil, light | 14.6 |
| | C12-15 Alcohols benzoate | 5 |
| | Safflower oil | 3 |
| | Sunflower oil | 3 |
| | Jojoba oil | 3 |
| | Fragrance | 2 |
| | Total | 100 |

*product has a creamy-like tactile feel.

### Example 3

### Cleansing Data

The formulations described in Tables 2-4 were individually tested for their ability to remove dirt, grease, and oil-based paint from skin. Dirt, grease, or oil-based paint was applied to skin. The formulations were applied to and rubbed on skin. Skin was rinsed with tap water. The dirt, grease, or oil-based paint was removed from the skin.

### Example 4

### Additional Assays that Can Be Used To Test Compositions

Additional testing to confirm the skin efficacy of the compositions of the present invention can be determined by methods known to those of ordinary skill in the art. The following are non-limiting assays that can be used in the context of the present invention. It should be recognized that other testing procedures can be used, including, for example, objective and subjective procedures.

Skin Exfoliation: The volar forearm skin can be patched with 5.0% dansyl chloride to achieve fullthickness SC staining within pre-determined treatment sites. Formulations can then be applied twice-daily for 5 days to stained sites. Fluorescence can be measured daily using a custom “Fluorescence Light Examiner” (FLX), a sensitive remittance fluorimeter. Fluorescence values can be used to calculate the first order decay kinetics of dansyl chloride washout (providing an index of SC exfoliation rate).

Skin Firmness and Elasticity Assay with a Hargens Ballistometer: Skin firmness and elasticity can be measured using a Hargens ballistometer, a device that evaluates the firmness and elasticity of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Skin Moisture/Hydration Assay: Skin moisture/hydration benefits can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72° C.). Three separate impedance readings can be taken on each side of the face, recorded, and averaged. The T5 setting can be used on the impedance meter which averages the impedance values of every five seconds application to the face. Changes can be reported with statistical variance and significance.

Skin Dryness, Surface Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman and Gams (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and they are of the replicas covered by wrinkles or fine lines was determined.

Surface Contour of the Skin Assay with a Profilometer/Stylus Method: The surface contour of the skin can be measured by using the profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Rₐ=Standardize roughness; lₘ=the traverse (scan) length; and y=the absolute value of the location of the profile relative to the mean profile height (x-axis).

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether a composition is inducing irritation. The measurements can be made on each side of the face and averaged, as left and right facial values. Skin clarity can also be measured using the Minolta Meter. The measurement is a combination of the a*, b, and L values of the Minolta Meter and is related to skin brightness, and correlates well with skin smoothness and hydration. Skin reading is taken as above. In one non-limiting aspect, skin clarity can be described as L/C where C is chroma and is defined as (a²+b²)^{1/2}

All of the skin-active ingredients, compositions, or methods disclosed and claimed in this specification can be made and executed without undue experimentation in light of the present disclosure. While the skin-active ingredients, compositions, or methods of this invention have been described in terms of particular embodiments, it will be apparent to those of skill in the art that variations may be applied to the skin-active ingredients, compositions, or methods and in the steps or in the sequence of steps of the method described herein without departing from the concept, spirit and scope of the invention.

### REFERENCES

Any one of the references identified in the specification, to the extent that they provide exemplary procedural or other details supplementary to those set forth in this specification, are specifically incorporated by reference.

1.-28. (canceled)

29. A method of treating skin, the method comprising topically applying to skin in need thereof a stable anhydrous dispersion, wherein topical application of the anhydrous dispersion treats the skin, and wherein the anhydrous dispersion consists essentially of:
(a) a dispersed sugar phase comprising granulated sugar having a mean aperture (MA) size between 200 μm to 1000 μm and powdered sugar having an average particle size between 30 μm to 150 μm, wherein the anhydrous dispersion comprises 55% to 75% by weight, based on the total weight of the anhydrous dispersion, of a combination of granulated sugar and powdered sugar; and
(b) a continuous oil phase, wherein the anhydrous dispersion comprises at least 20% by weight, based on the total weight of the anhydrous dispersion, of an oil or a mixture of oils,
wherein the weight ratio of granulated sugar to powdered sugar within the anhydrous dispersion is 1:1 to 2:1, and
wherein the anhydrous dispersion does not include a surfactant, an emulsifier, and a thickening agent.

30. The method of claim 29, wherein the anhydrous dispersion comprises:
60% to 70% by weight, based on the total weight of the anhydrous dispersion, of a combination of the granulated sugar and the powdered sugar; and
20% to 35% by weight, based on the total weight of the anhydrous dispersion, of the oil or the mixture of oils.

31. The method of claim 30, wherein the weight ratio of the granulated sugar to the powdered sugar is 1.3:1 to 2:1.

32. The method of claim 29, wherein the oil is mineral oil, a plant derived oil, or a triglyceride or a combination thereof.

33. The method of claim 32, wherein the oil is a plant derived oil selected from the group consisting of safflower oil, sweet almond oil, sunflower oil, jojoba oil, or olive or, or a combination thereof.

34. The method of claim 32, wherein the oil is mineral oil.

35. The method of claim 29, wherein the anhydrous dispersion does not include a triglyceride and a silicone-containing compound.

36. The method of 29, wherein the anhydrous dispersion moisturizes skin.

37. The method of claim 36, wherein the anhydrous dispersion is applied to dry, flaky, or cracked skin.

38. The method of claim 29, wherein the anhydrous dispersion remains stable when stored at 20° C. to 25° C.

39. The method of claim 29, wherein the anhydrous dispersion consists of the granulated sugar, the powdered sugar, and the oil or mixture of oils.

40. The method of claim 29, wherein the anhydrous dispersion further includes at least one plant extract.

41. The method of claim 29, wherein the anhydrous dispersion consists of the granulated sugar, the powdered sugar, the oil or mixture of oils, and the at least one plant extract.
